(19) United States
(12) Patent Application Publication
(10) Pub. No.: US 2003/0055435 A1
(43) Pub. Date: Mar. 20, 2003

(54) Orthopaedic implant shaper

(76) Inventor: Earl Frederick Barrick, McLean, VA (US)

Correspondence Address: Earl Frederick Barrick, 8904 Gallant Green Drive, McLean, VA, 22102-1515, US

(21) Appl. No.: 10/003,855

(22) Filed: Dec. 6, 2001

Related U.S. Application Data
(60) Provisional application No. 60/253,185, filed on Nov. 27, 2000.

(51) Int. Cl. A61F 2/00; A61F 2/32; A61F 2/34; A61F 2/36; A61F 2/38; A61B 17/58; A61B 17/60; G01B 11/24; G01B 11/30

(52) U.S. Cl. 606/102; 356/601

(57) ABSTRACT

This invention is a system consisting of a flexible fiber optic curvature sensor device, a computer, and an implant-shaping machine. The flexible fiber optic curvature sensor device is a tape used to determine the required three-dimensional shape of an orthopaedic implant. The sterilized tape is applied to the fractured bone in surgery once it is reduced. The shapes and sizes of various metal orthopaedic implants are stored on the hard disc of the computer in a lookup table file. The operator inputs the type of implant to be contoured and the number of holes of a plate or length of a rod. The digitized contour of the tape is then matched with the particular bone implant. The information is transmitted to the implant-shaping machine to program the settings for the actual contouring. The implant-shaping machine consists of a series of opposing hydraulic cylinders with dies arranged in rows on rocking platforms. Each unit consists of a pair of opposing hydraulic cylinders that work reciprocally to move dies in relation to the metal fixation implant.

### BACKGROUND OF THE INVENTION

This application claims the benefit of U.S. Provisional Application Ser. No. 60/253,185, filed Nov. 27, 2000.

The present invention relates to systems for shaping orthopaedic implants, specifically to shaping said implants using a computerized method of templating the needed shape and shaping with a semi-robotic machine.

Malleable metal plates and rods are used in orthopaedic surgery. Metal plates used for holding fractures must be contoured for application to the reduced bone. A template can be used to ascertain the shape of the bone onto which the plate is to be applied. The surgeon then manually contours the plate to match the template, using bending irons and/or a hand-operated bending tool that sits on a table. Other manually operated devices have been described. Metal rods are used in spinal surgery. Usually templates are not used to prepare to contour a rod; they are shaped manually by trial and error.

Using these methods, it is often difficult to match the implant to the curvatures of the involved bone. Multiple attempts are often needed, and sometimes a less than ideal final shape is accepted due to the difficulty of shaping the implant in six degrees.

Langlotz, et al., describe a computer-assisted method of measuring the contour of the bone using an image-guided system, digitizing the shape by taking multiple points with an optically tracked probe. An optical tracking system with a separate computer and a set of optical cameras on a stand are required. Multiple points must be taken from the site with a digitizing probe. This information is then transmitted to a computer workstation, which calculates the angles needed to contour a plate or rod. Another version uses an object scanner to obtain the contour parameters. The shape of the implant is contoured by the above-described hand-operated methods. The implant shape is calculated with the optical tracking system with light emitting diodes attached to the bending machine and compared with the computer model. This complex method only replaces the hand-formed template that is currently in widespread use, still requiring bending of the implant by hand.

Industrial plate or rod bending machines are generally designed to repetitively contour the metal to the same shape. Also they work with much larger plates, rods, or bars of metal. These machines often have means of moving the work piece past the bending elements.

### SUMMARY OF THE INVENTION

This invention is a system consisting of a flexible fiber optic curvature sensor device, a computer, and an implant-shaping machine. The flexible fiber optic curvature sensor device is a tape used to determine the required three-dimensional shape of an orthopaedic implant. The sterilized tape is applied to the fractured bone in surgery once it is reduced. The computer software program analyzes the data from the electric changes in the fiber optics as the tape is flexed. The computer calculates the shape of the tape in six degrees of position to transmit to the implant-shaping machine. In one version, the shape is displayed as a computer graphic. This sensor relies on linear, bipolar modulation of light throughput in specially treated fiber optic loops sealed in laminations. The sensor consists of paired loops of optical fibers that have been treated on one side to lose light proportional to bending. The lost light is contained in absorptive layers that prevent the interaction of light with the environment. The interface box illuminates the loops, detects return light, and relays information to the computer having the software that calculates the shape of the sensor.

The shapes and sizes of various metal orthopaedic implants are stored on the hard disc of the computer in a lookup table file. The operator inputs the type of implant to be contoured and the number of holes of a plate or length of a rod. The specifications in the lookup table include the length, width, and depth of each implant. The digitized contour of the tape is then matched with the particular bone implant. One end of the tape is designated as the starting point to determine the length of the implant. The information is transmitted to the implant-shaping machine to program the settings for the actual contouring.

The implant-shaping machine consists of a series of opposing hydraulic cylinders with dies arranged in rows on rocking platforms. Each unit consists of a pair of opposing hydraulic cylinders that work reciprocally to move dies in relation to the metal fixation implant. The implant is placed in the space between the series of dies.

The digitized shape of the bone implant is used to set the dies in the implant-shaping machine. The dies are set for the shape and length of the virtual implant as programmed from the computer data. There are two rows of hydraulic cylinders that move the dies into position to contour the implant. The hydraulic cylinders come together beyond the predetermined length of the implant. The dies are driven by the rows of hydraulic cylinders to bend the implant in one plane. Twisting or contouring in a rolling plane is accomplished by having the opposing cylinder-die units rotate on a rotating platform powered with individual cylinders. These twisting cylinders have gimbals, or other rotating means, at each end to accommodate for the angles that develop as the platform holding the opposing cylinders rotates in a seesaw fashion. Electrical actuators power the hydraulic cylinder-dies and rotating cylinders. Position sensors determine the relative positioning of the cylinders in relation to one another.

The machine adapts to curved implants by having the axis of the rotating platform assume the predetermined contour of a standard curved implant. This change in the shape of the axis is accomplished by having the axis move to a base of this shape. The axis is supported by a flexible narrow band that is moved by a series of cylinders. Another version is especially designed for curved implants.

One version has stacked cylinders to move the dies with sufficient force to bend stronger bone implants. Dual rotating cylinders are used in another version to twist stronger implants.

Another version uses a malleable light metal template that is contoured by hand, to ascertain the desired shape for the bone implant, the method now in common practice. However, a scanner then measures the contours and a computer conveys this shape to the implant-shaping machine.

### BRIEF DESCRIPTION OF THE DRAWINGS

FIG. 1. Illustration of the fiber optic curvature sensor tape lying against the bone.

FIG. 2. Illustration of shaped plates lying across fractures.

FIG. 3. Illustration of the implant-shaping machine at rest.

FIG. 4. Cross-section of the implant-shaping machine.

FIG. 5. Illustration of the implant-shaping machine activated.

### DETAILED DESCRIPTION OF THE PREFERRED EMBODIMENTS

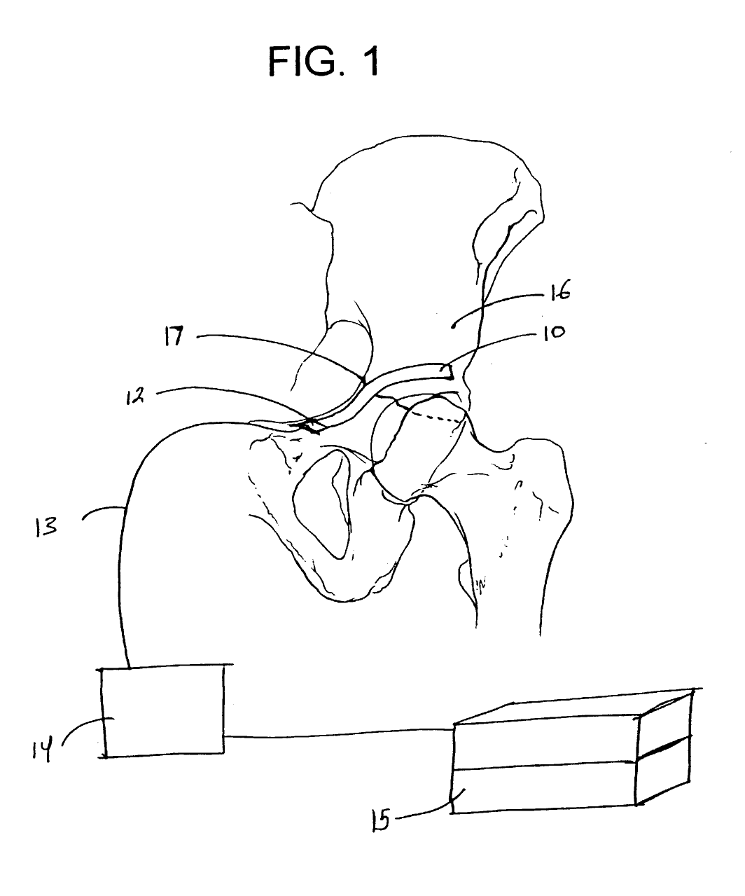

The first step in using this system is shown in the example in FIG. 1. The fiber optic curvature sensor tape 10 is placed on innominate bone 16 after fracture 17 has been reduced. Interface box 12 illuminates the loops and detects return light in fiber optic curvature sensor tape 10 and relays information to computer 14. Computer 14 calculates the shape of the sensor tape and transmits this data to implant-shaping machine 15.

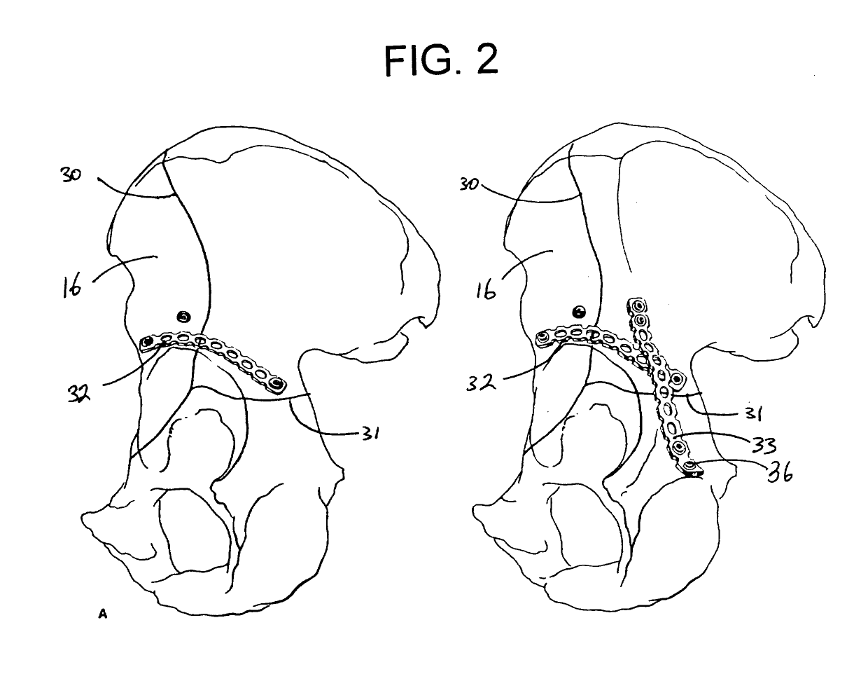

Innominate bone 16 is shown in FIG. 2A with shaped plate 32 lying across fracture 30. Innominate bone 16 in FIG. 2B has a second shaped plate 33 over second fracture line 31. Screws 36 are holding shaped plates 32 and 33 against innominate bone 16.

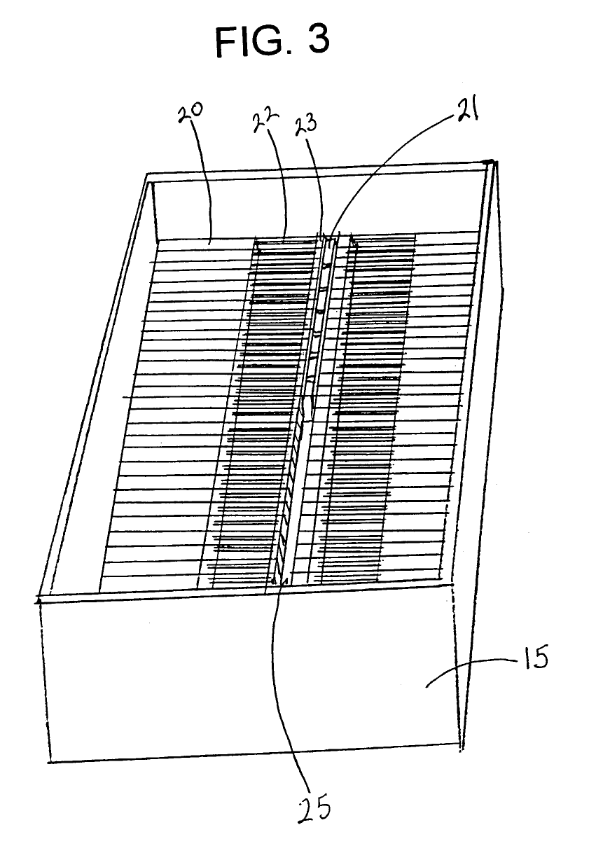

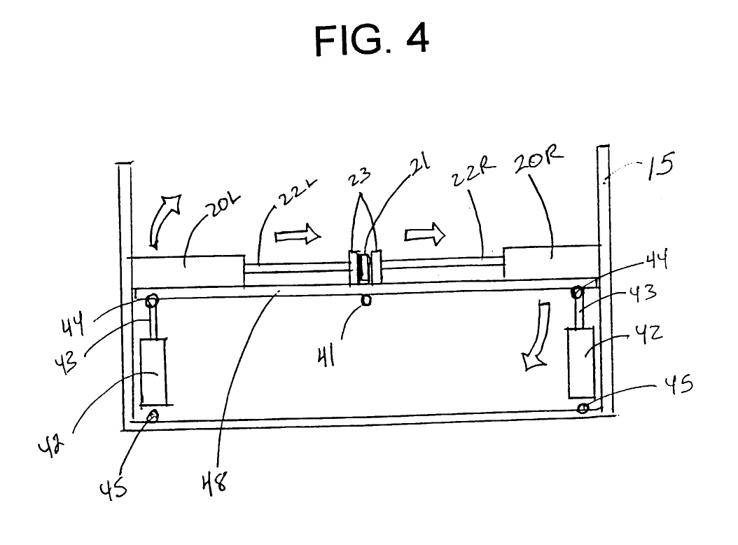

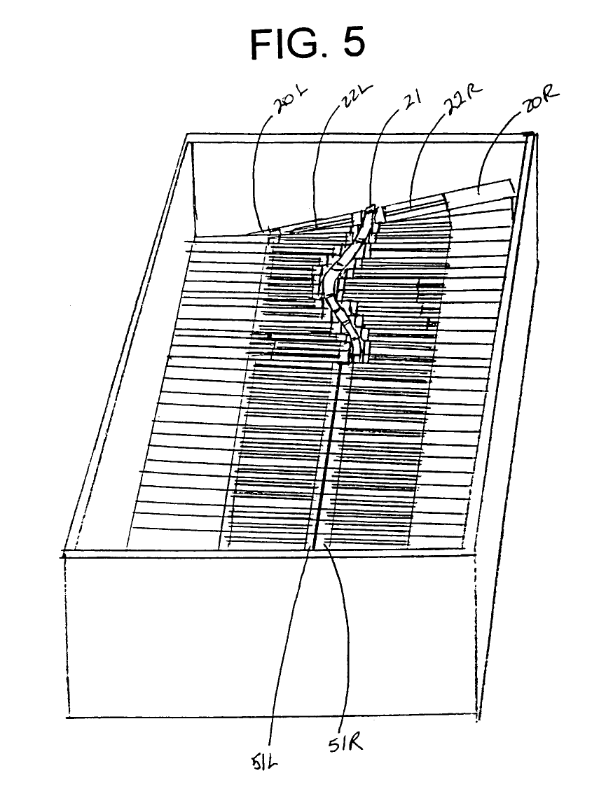

Implant-shaping machine 15 is dormant in FIG. 3. Cylinders 20 are seen in series on each side of space 25 into which bone plate 21 is placed for shaping. Cylinders 20 are on platform 48 that can rotate on axis 41 in a seesaw fashion, as seen in FIG. 4. Plate 21 is placed between the opposing series on cylinders 20. Cylinders 20 are activated by instructions from computer 14 to shape plate 21. The mechanism consists of cylinder 20 driving rod 22 to move die 23 against plate 21. As left cylinder 20L moves rod 22L and die 23 forward, right cylinder 20R with rod 22R and die 23 draws back to bend plate 21, as illustrated in FIGS. 4 and 5. Bending action is programmed to occur at the level of plate 21. Where plate 21 ends, the series of cylinders 20, rods 22 and dies 23 come together, keeping plate 21 from migrating. If a twist is needed in plate 21, cylinders 42L and 42R move rods 43L and 43R reciprocally to move platform 48 on pivot 41. Gimbals 44 and 45 permit rotation between cylinder 42 and rod 43 combination. Once contoured plate 21 is removed and laid against innominate bone 16 to secure fracture 17, 30 or 31 once screws 36 are inserted.

While the invention has been described with reference to specific embodiments, modifications and variations of the invention may be construed without departing from the scope of the invention, which is defined in the following claims.

I claim:

1. A system for custom contouring or shaping an orthopaedic implant, such as a metal plate or rod, comprising a flexible fiber optic curvature sensor to template the preferred shape of the implant and transmit the shape electronically to a computer, a computer that has a look-up table on its hard disc and that is programmed to transmit the shape to a control device, and an implant shaping apparatus that is controlled by the information derived from the flexible fiber optic curvature sensor device and that shapes an implant with a plurality of hydraulic cylinders arranged to contour said implant.

2. The system of claim 1, wherein the flexible fiber optic curvature sensor consists of paired loops of optical fibers that are in a flexible tape and that have been treated on one side to lose light proportional to bending, in which the lost light is contained in absorptive layers that prevent the interaction of light with the environment, and has an interface box that illuminates the loops, detects return light, and relays information to a computer having the software that calculates the shape of the sensor.

3. The system of claim 1, wherein the computer is programmed to transmit the implant shape data to a control device that directs the implant shaping apparatus to shape the implant to match the shape derived from the flexible fiber-optic sensor.

4. The system of claim 1, wherein the implant shaping apparatus is comprised of a plurality of hydraulic cylinders arranged to contour an implant under the direction of the control device.

5. A flexible fiber optic curvature sensor, that can be sterilized, to template the preferred shape of an implant and transmit the shape electronically to a computer that transmits the data to a control device that operates any other implant shaping apparatus.

6. An implant shaping apparatus comprised of a plurality of hydraulic cylinders arranged to contour an implant under the direction of a control device, in which the desired shape is determined by a three-dimensional scanner that scans a hand-contoured template of the shape of the bone.

7. An implant shaping apparatus comprised of a plurality of hydraulic cylinders arranged to contour an implant under the direction of a control device, in which the desired shape is determined by any means.

8. A method of contouring an orthopaedic implant comprising of the steps of determining the desired shape of said implant using a sterilized flexible fiber optic curvature sensor by laying it against the exposed bone during a surgical operation, using the data there from in a computer to digitize the information, providing a control device that uses this data to control an implant shaping apparatus, placing an implant in the implant shaping apparatus, activating this implant shaping apparatus to contour the implant, said implant shaping apparatus being comprised of a plurality of hydraulic cylinders arranged to contour an implant under the direction of the control device.
